(19) United States
(12) Patent Application Publication
(10) Pub. No.: US 2025/0161209 A1
(43) Pub. Date: May 22, 2025

(54) SYSTEM AND METHOD FOR AEROBIC RESPIRATORY TREATMENT

(71) Applicant: Jeff Braile, Boca Raton, FL (US)

(72) Inventor: Jeff Braile, Boca Raton, FL (US)

(21) Appl. No.: 19/034,337

(22) Filed: Jan. 22, 2025

Related U.S. Application Data
(63) Continuation-in-part of application No. 18/378,020, filed on Oct. 9, 2023.
Application No. 18/378,020 is a continuation-in-part of application No. 17/554,057, filed on Dec. 17, 2021, now Pat. No. 11,779,621.

(51) Int. Cl. A61K 9/00; A61K 9/50; A61K 31/00

(52) U.S. Cl. CPC A61K 9/0073; A61K 9/5089; A61K 31/658

(57) ABSTRACT

A method including the steps of extracting and producing a distillate that includes one or more oil-based cannabinoids and/or nutraceuticals; melting the distillate and combining the melted distillate with pharmaceutical or similar quality salt particles to cause the distillate to coat the particles of salt; grinding the coated salt particles to produce a compound particle mixture of salt and distillate; repeating the steps of baking and further grinding until the compound particle mixture reaches a desired consistency; and releasing the ground compound particle mixture into an air environment for inhalation into the lungs.

### BACKGROUND OF THE INVENTION

This non-provisional patent application is a Continuation-In-Part (CIP) of co-pending non-provisional patent application Ser. No. 18/378,020 filed on Oct. 9, 2023, which is a Continuation-In-Part (CIP) of non-provisional patent application Ser. No. 17/554,057 filed on December 17, 2021, now U.S. Pat. No. 11,779,621 issued Oct. 10, 2023.

### FIELD OF THE INVENTION

The present invention relates to a method of producing oil-based compositions, including oil-based cannabinoids and nutraceuticals, combined with salt.

### DESCRIPTION OF THE RELATED ART

Respiratory therapy involving the inhalation of pharmaceutical salt, or similar quality salt, has been used for decades as an anti-inflammatory for the respiratory channels to aid in healthy breathing and overall good health. This is due to the fact that the body absorbs sodium nearly completely because of how soluble salts are in the body and digestive tract. For example, athletes use salt tablets to quickly recover and rejuvenate during contests or vigorous workouts. According to the National Institute of Health (NIH), the lungs are a particularly attractive target for the delivery of beneficial therapies in the treatment of respiratory diseases, while providing an enormous surface area and a relatively low enzymatic controlled environment for systematic absorption of medications. Furthermore, CBD isolate crystals and other crystallized cannabinoids and oil-based nutraceuticals combined with salt (e.g., pharmaceutical grade salt) and released into the environment to be inhaled into the lungs and sinuses is the most efficient way of ingesting these oil-based compounds into the body.

CBD, which has been shown to help the respiratory process, while providing additional benefits of pain relief, reduced anxiety and a number of other purported attributes has a number of issues with regards to use by the body. In particular, CBD is both fat and water soluble and it is difficult for the body to utilize. Recent research and data show that at most, only 13-19% of consumed CBD is absorbed by the body. Further, on average, only 5-10% CBD that users take orally is ultimately absorbed. By combining salt with CBD or other oil-based cannabinoid compounds, as well as other oil-based compounds and nutraceuticals, for inhalation into the lungs, the ability for the body to absorb these oil-based compounds is greatly enhanced.

The oil-based CBD isolate or other oil-based cannabinoid compound used in the invention has a delta-9 tetrahydrocannabinol [THC] concentration of not more than 0.3 percent on a dry weight basis or such other limit permitted under US federal law. And in at least one embodiment of the invention, the oil-based CBD isolate or other oil-based cannabinoid compound has a zero concentration of THC (i.e., THC free). The invention also contemplates use of oil-based CBD isolate and other oil-based cannabinoid compounds having a delta-9 tetrahydrocannabinol [THC] concentration of higher than 0.3 percent on a dry weight basis if permitted by U.S. federal law after the filing date of this patent application.

### SUMMARY OF THE INVENTION

According to one embodiment of the present invention, particles of oil-based CBD isolate may be combined with particles of pharmaceutical grade salt which together are released into the air environment to be inhaled into the lungs and sinuses. Once inhaled, these oil-based CBD isolate particles and salt particles absorb irritants, including allergens and toxins, from the respiratory system. This helps to break up mucus and reduce inflammation, resulting in clear airways.

When salt is combined with CBD, in particle form for inhalation into the lungs, a multiplier effect is created due to the benefits of the CBD. More particularly, the compound created by the mixture of oil-based CBD and salt provides multiple benefits for both respiratory health as well as mental wellbeing. The combination of salt and oil-based CBD may be ground into particles by a generator apparatus or handheld generator device and released into the air environment for direct inhalation through the nose and/or mouth and into the respiratory channels and lungs.

In a further embodiment of the invention, the oil-based CBD and salt combination may be miniaturized into particles and then aerosolized through a handheld nebulizer or canister designed to propel the particles toward the nose and mouth where it can enter the nasal cavity, respiratory channels and lungs.

In one embodiment of the invention, cannabis is extracted from the cannabis plant using a solvent (e.g., butane, ethanol or other solvent) to yield a liquid. This liquid is then evaporated to produce a concentrated oily residue distillate. This oily distillate contains the compounds of the hemp plant and can be processed and refined further to include a variety of oils including full spectrum, broad spectrum, or any particular oils containing CBD, CBG, CBN or other oils in the hemp plant. The oily distillate may also be other oil-based nutraceuticals not found in the cannabis plant, such as, but not limited to, NAD, Glutathione, oil-based vitamin supplements, fish oil, and testosterone. The distillate is then melted and combined with a pharmaceutical or similar quality salt which has been previously heated to aid in cohesion. The salt preferably has a particle size of 80 microns or smaller. Next, the oily distillate is baked on to the salt, so that the distillate (e.g., CBD, CBG, CBN or other oil-based nutraceuticals) essentially coats the salt particles. The resultant combination is further ground to produce a particle mixture of the distillate and salt. This process of grinding, baking and further grinding is then repeated until the compound reaches the desired consistency. This allows for a more consistent ratio of salt to distillate in the resultant ground distillate/salt compound mixture. The resulting compound mixture is then used in either a halo generator or handheld device which propels miniaturized or chopped particles of the distillate/salt mixture into the air where the particles can be inhaled. In one embodiment, the distillate is CBD. In another embodiment, the distillate is NAD or Glutathione. In a further embodiment, the distillate is an oil-based vitamin composition. In still a further embodiment, the distillate is a nutraceutical composition. In still a further embodiment, the distillate is testosterone.

In yet a further embodiment, the distillate is cannabis oil or extract.

### BRIEF DESCRIPTION OF THE DRAWINGS

For a fuller understanding of the nature of the present invention, reference should be had to the following detailed description taken in connection with the accompanying drawing in which:

FIG. 1 is a flow chart, in schematic form, illustrating the sequence of steps of the method of the present invention according to one embodiment thereof;

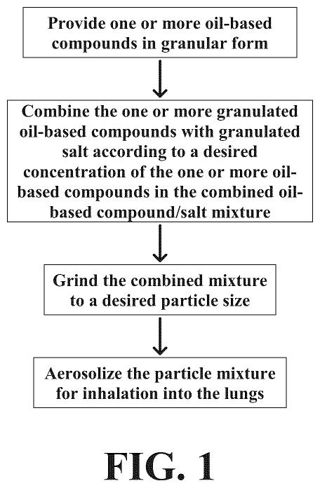

FIG. 2 is a general diagram showing the main components of a generator device in accordance with one embodiment for grinding oil-based composition particles in combination with pharmaceutical grade salt particles and releasing the particles into the surrounding air atmosphere where they can be inhaled directly through the nose and mouth and into the lungs;

FIG. 3 is a perspective view of a handheld generator device for grinding oil-based composition particles and pharmaceutical grade salt particles, and releasing the particles for direct inhalation by the user through the nose and/or mouth and into the lungs, in accordance with one embodiment of the invention;

FIG. 4 is a flow chart, in schematic form, illustrating the sequence of steps of the method of the present invention in accordance with one embodiment of the invention; and

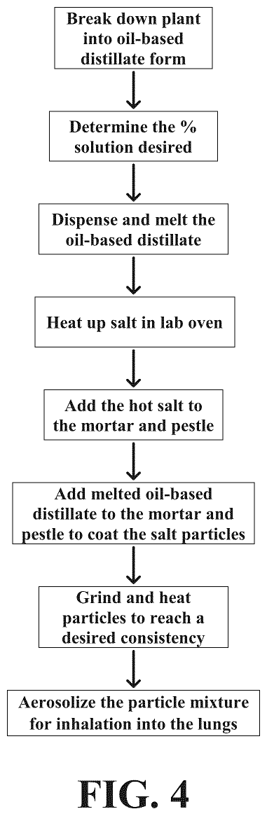

FIG. 5 is a flow chart, in schematic form, illustrating the sequence of steps of the method of the present invention according to yet a further embodiment of the invention.

Like reference numerals refer to like parts throughout the several views of the drawings.

### DETAILED DESCRIPTION OF THE PREFERRED EMBODIMENTS

The present invention provides a method of producing an oil-based composition, in particle form and a method of delivering particles of the oil-based composition into an air environment to be inhaled deep into the lungs and sinuses for therapeutic benefits. The present invention contemplates the use of oil-based CBD isolate and all cannabinoids, such as, for example, CBG and CBN, as well as cannabis oil or extract, fish oil, oil-based vitamin supplements, testosterone and nutraceutical compositions combined with salt particles for inhalation into the lungs and sinuses, thereby providing the desired therapeutic benefits to increase respiratory health, body function and overall health.

As previously noted, the inhalation of pharmaceutical grade salt particles has been used for decades as an anti-inflammatory for the respiratory channels. The present invention proposes to combine pharmaceutical grade salt particles with the pure oil-based CBD isolate particles to be released into the air for inhalation into the lungs, sinuses and respiratory channels for purposes of respiratory health and enhanced mental clarity. According to one embodiment of the present invention, CBD isolate particles are present in an amount of between 0.5% and 55% by weight of the composition and particles of pharmaceutical grade salt are present in an amount of between 45% and 99.5% by weight of the composition.

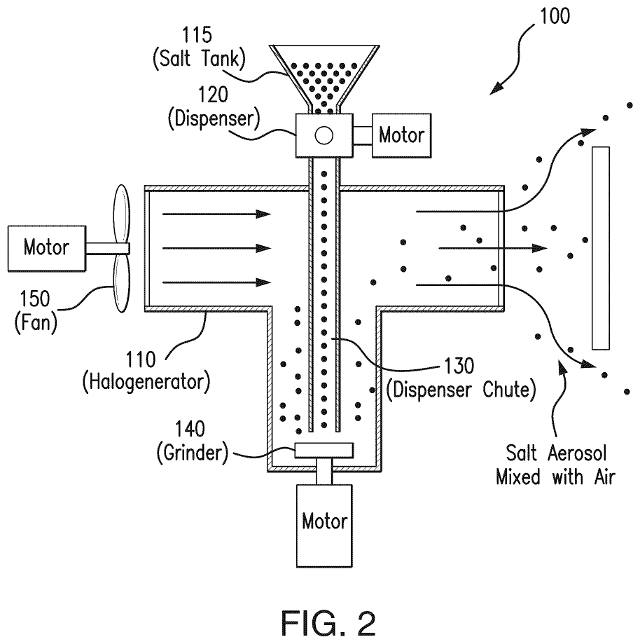

Referring to FIG. 2, a general diagram of a generator apparatus is shown for miniaturizing the oil-based CBD isolate or other oil-based composition into precise sized particles, including microscopic particles ranging between 1 and 40 microns in size and macroscopic particles ranging between 41 and 100 microns in size. As noted above, pharmaceutical grade salt is combined with the oil-based CBD isolate or other oil-based composition and is also miniaturized to the precise size of between 1 and 100 microns. The generator apparatus is particularly in the form of a halo generator 110 that includes a hopper or salt tank 115 within which is fed the combined oil-based composition and salt (e.g., pharmaceutical grade salt). A motorized dispenser 120 releases the particles of oil-based composition and pharmaceutical grade salt through a dispenser shoot 130 where the particles are then further grinded by a motorized grinder 140 having a blade mechanism that accurately grinds and/or cuts the particles into the desired particle size of between 1 and 100 microns. A motorized fan 150 blows the particle mixture out from the opposite side of the halo generator, to release the particles of oil-based composition and salt into the air atmosphere for direct inhalation into the lungs and sinuses. It is noted that other oil-based cannabinoid isolates, such as, for example, CBG and/or CBN, may be used in the generator apparatus 100 to deliver the cannabinoid isolate particles and salt particles in a suspended form in the air environment for inhalation into the lungs and sinuses, to thereby provide the desired therapeutic benefits.

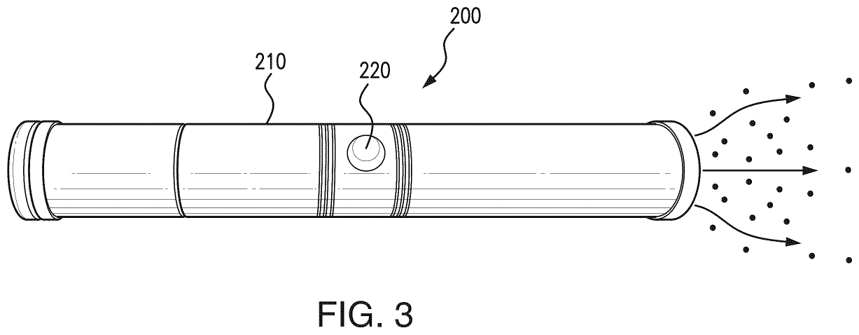

FIG. 3 illustrates a handheld generator device 200 that is used to grind and release the oil-based cannabinoid particles or other oil-based compositions for inhalation through the nose and/or mouth of a user. Similar to the generator apparatus of FIG. 2, the oil-based cannabinoid isolate particles, such as crystallized CBD isolate particles, or other oil-based composition particles are combined with pharmaceutical grade salt and ground within the handheld device 200 for releasing the particles in a desired particle size for inhalation by the user. The handheld device 200 is preferably in the form of an elongate tube 210 that contains the motorized blower, grinder and chamber for holding the oil-based composition particles and pharmaceutical grade salt crystals.

Operation of a button 220 or other actuator mechanism serves to acuate the grinder and the blower within the handheld device 200 to release the particles, as shown in FIG. 3.

In one embodiment of the invention, cannabis is extracted from the cannabis plant using a solvent (e.g., butane, ethanol or other solvent) to yield a liquid. This liquid is then evaporated to produce a concentrated oily residue distillate. This distillate contains the compounds of the hemp plant and can be processed and refined further to include a variety of oils including full spectrum, broad spectrum, or any particular oils containing CBD, CBG, CBN, cannabis oil, cannabis extract, or other oils in the hemp plant. The distillate may also be other oil-based nutraceuticals not found in the cannabis plant, such as, but not limited to, NAD, Glutathione, testosterone, fish oil and other oil-based vitamin supplements. The distillate is then melted and combined with a pharmaceutical or similar quality salt which has been previously heated to aid in cohesion. The salt and distillate combination is then grinded to produce crystals or small particles. Next, the distillate is baked into the salt, so that the salt essentially coats the distillate particles (e.g., CBD, CBG, CBN or other oil-based nutraceuticals). The resultant combination is further ground to produce a particle mixture of the distillate and salt. This process of grinding, baking and further grinding is then repeated until the compound reaches the desired consistency. This allows for a more consistent ratio of salt to distillate in the resultant ground distillate/salt compound. The resulting compound is then used in either a halo generator 110 or handheld generator device 200 which propels miniaturized or chopped particles of the distillate/salt compound into the air where the particles can be inhaled. In one embodiment, the distillate is CBD. In another embodiment, the distillate is CBG or CBN. In another embodiment, the distillate is NAD or Glutathione. In a further embodiment, the distillate is an oil-based vitamin composition. In still a further embodiment, the distillate is a nutraceutical composition. In a further embodiment, the distillate is testosterone. In yet a further embodiment, the distillate is cannabis oil or extract.

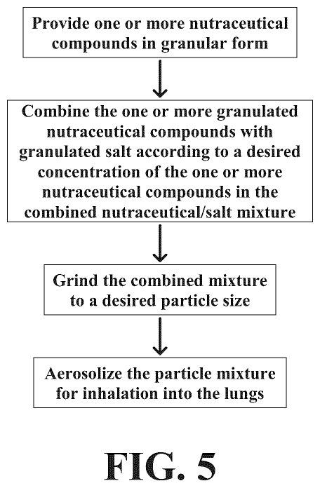

Referring to FIG. 5, a further embodiment of the invention is illustrated in accordance with a series of steps. More specifically, the embodiment of FIG. 5 includes providing one or more nutraceutical compounds in granular form and combining the one or more granulated nutraceutical compounds with granulated salt according to a desired concentration of the one or more nutraceutical compounds in the combined mixture of the nutraceutical compounds and salt.

Next, the combined mixture of the one or more granulated nutraceutical compounds and salt are ground to a desired particle size. The final step of the process is aerosolizing the ground particle mixture of the one or more nutraceutical compounds and salt for inhalation into the lungs of a human. It is noted that the method according to this embodiment may include combining any number of nutraceutical compounds with salt and grinding the mixture of the combined nutraceutical compounds and salt to achieve the desired particle size for subsequent aerosolization for inhalation into the lungs of a human. As noted in the previous embodiments, the salt may be a pharmaceutical grade salt or similar quality salt.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A method of producing an oil-based particle composition comprising the steps of:
a) producing a distillate containing an oil-based compound;
b) grinding at least the distillate to produce distillate particles;
c) combining the distillate with salt particles;
d) baking the particles of salt and distillate to cause the distillate to coat the particles of salt; and
e) grinding the coated particles of salt to produce a particle mixture of the distillate and salt.

2. The method of claim 1 wherein the ground particle mixture of the distillate and salt is baked and subsequently ground to yield a second particle mixture of the distillate and salt.

3. The method of claim 1 further comprising the step of releasing the particle mixture of the distillate and salt into the environment for inhalation into the lungs of a human.

4. The method of claim 2 further comprising the step of releasing the particles of the second ground mixture of the distillate and salt into the environment for inhalation into the lungs of a human.

5. The method of claim 1 wherein the distillate is pure cannabidiol isolate.

6. The method of claim 1 wherein the distillate is CBG.

7. The method of claim 1 wherein the distillate is CBN.

8. The method of claim 1 wherein the distillate is NAD.

9. The method of claim 1 wherein the distillate is Glutathione.

10. The method of claim 1 wherein the distillate is an oil-based vitamin supplement.

11. The method of claim 1 wherein the distillate is fish oil.

12. The method of claim 1 wherein the distillate is testosterone.

13. The method of claim 1 wherein the distillate is cannabis oil or cannabis extract.

14. A method of producing a particle composition comprising the steps of:
a) providing at least one nutraceutical compound in granular form;
b) combining the at least one granulated nutraceutical compound with granulated salt; and
c) grinding the combination of the at least one granulated nutraceutical compound and granulated salt to produce a ground mixture of particles containing the at least one nutraceutical compound and the salt.

15. The method of claim 14 further comprising the step of releasing the mixture of the at least one nutraceutical particles and salt particles into the environment for inhalation into the lungs of a human.

16. The method of claim 14 further comprising the steps of:
a) providing a plurality of different nutraceutical compounds in granular form;
b) combining the plurality of different granulated nutraceutical compounds with granulated salt according to a desired concentration of the plurality of different nutraceutical compounds in the combined mixture of the plurality of nutraceutical compounds and the salt; and
c) grinding the combined mixture to a desired particle size.

17. The method of claim 16 further comprising the step of aerosolizing the ground particle mixture for inhalation into the lungs of a human.

18. A method of producing a particle composition comprising the steps of:
a) providing at least one oil-based compound in granular form;
b) combining the at least one granulated oil-based compound with granulated salt; and
c) grinding the combination of the at least one granulated oil-based compound and granulated salt to produce a ground mixture of particles containing the at least one oil-based compound and the salt.

19. The method of claim 18 further comprising the step of aerosolizing the ground particle mixture for inhalation into the lungs of a human.
